(19) United States
(12) Patent Application Publication
(10) Pub. No.: US 2024/0271150 A1
(43) Pub. Date: Aug. 15, 2024

(54) AGRONOMIC PRACTICES AND SUSTAINABILITY METHODS INVOLVING YIELD TRAITS

(71) Applicant: PIONEER HI-BRED INTERNATIONAL, INC., JOHNSTON, IA (US)

(72) Inventors: JAMES W. BING, GRIMES, IA (US); JEFFREY HABBEN, URBANDALE, IA (US); JEFFREY R SCHUSSLER, MARION, IA (US); BENJAMIN P WEERS, POLK CITY, IA (US)

(73) Assignee: PIONEER HI-BRED INTERNATIONAL, INC., JOHNSTON, IA (US)

(21) Appl. No.: 18/567,214

(22) PCT Filed: Jun. 14, 2022
(86) PCT No.: PCT/US2022/033408

§ 371 Date: Dec. 5, 2023

Related U.S. Application Data
(60) Provisional application No. 63/210,521, filed on Jun. 15, 2021.

(51) Int. Cl. C12N 15/82; C07K 14/415

(52) U.S. Cl. CPC C12N 15/8261; C07K 14/415

(57) ABSTRACT

The compositions and methods relate to sustainable agronomic practices of corn plants expressing a transgenic yield trait (e.g., increased and extended expression of AG099 or a MADS-box polypeptide) are disclosed. These include application of nitrogen stabilizers, cover crops, and farming practices.

### FIELD

Embodiments disclosed herein relate to the field of plant molecular biology and agronomic traits.

### BACKGROUND

Corn is an agriculturally important crop and serves as a food and feed source for animal, human, and industrial uses. Increased grain yield may be achieved in maize plants by a variety of ways, including expression of a transgene to increase grain yield in addition to improved breeding. Performance of a transgene in a plant including the agronomic parameters, may be impacted by a variety of factors such as the use of expression elements including promoter/regulatory elements, the genomic location of the insert sequence, copy number of the inserted transgene and genetic (germplasm) and environmental factors such as soil, temperature, light and moisture. The identification of constructs, testing of orthologs and transformation events that result in increased grain yield of a maize plant at a commercially relevant level in the field are the result of a substantial and significant developmental effort towards product advancement. Accordingly, it would be desirable to have maize plants that demonstrate increased grain yield.

### SUMMARY

A method of optimizing exogenously applied nitrogen use in a field by a population of crop plants contain a transgenic trait affecting yield, the method includes providing crop plants, wherein the expression of a transgenic trait increases one or more yield related agronomic parameters, to a field that comprises applied nitrogen fertilizer and a nitrogen stabilizer; and increasing the nitrogen utilization rate and/or nitrogen assimilate rate during the crop plants growing season compared to a control population of plants not comprising the trait. In an embodiment, the plants are corn plants that comprise a heterologous polynucleotide that encodes a MADS-box polypeptide. In an embodiment, the corn plants are planted in one or more zones within the field, where the zones are characterized by nitrogen run-offs. In an embodiment, the applied nitrogen is about 10% to about 50% less per acre compared to a normal field not comprising the crop plants having the transgenic trait.

In an embodiment, the applied nitrogen is used more effectively by the crop plants as measured by increase in stalk and/or leaf nitrogen content compared to a control population of plants not comprising the trait. In an embodiment, the applied nitrogen is used more effectively by the crop plants as measured by increase in root length and/or root density compared to a control population of plants not comprising the trait. In an embodiment, the applied nitrogen is used more effectively by the crop plants as measured by increase in shoot and/or root biomass compared to a control population of plants not comprising the trait. In an embodiment, the plants are corn plants that comprise event DP-202216-6. In an embodiment, the crop plants are heat tolerant. In an embodiment, the cumulative applied nitrogen in a crop growing season is about 5% to about 50% less than an application rate of about 50 lbs to about 400 lbs of nitrogen per acre compared to a normal field not comprising the crop plants having the transgenic trait.

A method of rotating crops in one or more consecutive growing seasons, the method includes growing a population of transgenic corn plants in a first growing season in a field, wherein the expression of a polynucleotide that encodes a MADS-box polypeptide is increased due to a genetic modification, compared to a control plant; growing a population of a second crop plants in a second growing season in the field, wherein the second growing season is consecutive to the first growing season and wherein the second crop plant is not corn; and increasing yield of the population of the second crop plants compared to a control population of second crop plants grown in a control field. In an embodiment, the corn plants comprise event DP-202216-6. In an embodiment, the population of second crop plants is selected from the group consisting of soybeans, cotton, wheat and sorghum. In an embodiment, the increase in yield in the population of the second crop plants is due to increased sequestration of soil organic matter and/or nitrogen. In an embodiment, the corn plants have early maturity rating and are planted early during the first growing season such that the population of the second crop plants are planted early in the second growing season, the first and second growing seasons are complete within about 8-12 months. In an embodiment, the field is not tilled, practice conservation tilling, minimum tilling or strip till before planting the corn plants. In an embodiment, the corn plants are selected to be in a relative maturity zone 80 CRM to about 120 CRM and wherein the second crop plants are soybean plants selected to be in a relative maturity zone of Group 2 to Group 6.

In an embodiment, the field is planted with a cover crop prior to or during or after planting of the corn plants. In an embodiment, the field is planted with a soybean crop prior to planting of the first population of corn plants and then followed by the second crop comprising soybean plants, wherein the first population of corn plants and the second crop of soybean plants are plant in consecutive growing seasons. In an embodiment, the cover crop is a perennial cover crop that remains dormant during the crop growing season. In an embodiment, the cover crop is planted in a field that is strip tilled.

A method of increasing yield by minimizing plant-to-plant variability within a row of a population of corn plants grown in a field, the method includes providing a population of corn plants, wherein the expression of a polynucleotide that encodes a polypeptide comprising an amino acid sequence that is at least 90% identical to maize MADS box transcription factor (Zmm28) is increased due to a genetic modification compared to a control plant; planting the corn plants at a density of about 30,000-60,000 plants per acre wherein the row spacing is about 8 inches to about 36 inches; and growing the population of plants in a crop growing environment, wherein the average height variation of the corn plants within a row is less than about 5% to about 20% when measure at a vegetative growth state between V6 to about R1. In an embodiment, the corn plants comprise event DP-202216-6. In an embodiment, the corn plants comprise canopy architecture that does not vary by more than about 5% to about 10% when measured by average total surface area of the leaves. In an embodiment, the corn plants exhibit uniform standability when compared to a control population of corn plants. In an embodiment, the yield is increased due to synchronized pollination and/or increased silk emergence under drought stress.

A method for introgressing one or more transgenic insect control traits in corn plants, the method includes providing a first female inbred corn plant comprising event DP-202216-6 DNA and a first insect control transgenic trait, wherein the first insect control transgenic trait exhibits reduced inbred yield when the first insect control trait is present in a first population of corn plants in the absence of the event DP-202216-6; providing a first male inbred corn plant comprising a second insect control trait, wherein the second insect control transgenic trait exhibits reduced inbred yield when the second insect control trait is present in a second population of corn plants in the absence of the event DP-202216-6; and crossing the first female inbred plant and the first male corn inbred plant to produce hybrid corn plants comprising the first and second insect control traits and event DP-202216-6. In an embodiment, the first insect control trait in the first male inbred plant protects the corn plants from one or more lepidopteran pests. In an embodiment, the second insect control trait in the first female inbred corn plant protects the corn plants from one or more coleopteran pests.

A method of increasing and/or improving corn silage quantity and/or quality, the method comprising providing a population of corn plants, wherein the expression of a polynucleotide that encodes a polypeptide comprising an amino acid sequence that is at least 90% identical to SEQ ID NO: 1 is increased due to a genetic modification compared to a population of control plants; growing the population of plants in a crop growing environment in a field, wherein the harvested corn for silage exhibits an improved characteristic of increased silage yield, digestibility, or fermentability compared to the population of control plants. In an embodiment, the corn plants comprise event DP-202216-6. In an embodiment, the corn plants comprise one or more brown mid-rib (BMR) traits. In an embodiment, the corn plants exhibit improved silage quality when harvested earlier than a population of a control population of corn plants that exhibit the same or substantially similar comparative relative maturity (CRM) growth stage.

### DETAILED DESCRIPTION

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

The disclosure and content of International Patent Application Publication No. WO2019055141A (application serial number PCT/US2018/044498), as it relates to stature modification, are herein incorporated by reference in its entirety. In some embodiments, the event DP-202216-6 comprises a recombinant DNA construct and wherein the event DP-202216-6 comprises s a polypeptide that is at least 95% identical to SEQ ID NO: 1 disclosed in US20190320607, which AG099 polypeptide sequence and event DP-202216-6 are hereby incorporated by reference in their entirety.

Compositions of this disclosure include a representative sample of seeds which was deposited as Patent Deposit No. PTA-124653 and plants, plant cells, and seed derived therefrom. Applicant(s) have made a deposit of at least 2500 seeds of maize event DP-202216-6 (Patent Deposit No. PTA-124653) with the American Type Culture Collection (ATCC), Manassas, VA 20110-2209 USA, on Jan. 12, 2018. These deposits will be maintained under the terms of the Budapest Treaty on the International Recognition of the Deposit of Microorganisms for the Purposes of Patent Procedure. The seeds deposited with the ATCC on Jan. 12, 2018 were taken from a representative sample deposit maintained by Pioneer Hi-Bred International, Inc., 7250 NW 62^{nd} Avenue, Johnston, Iowa 50131-1000. Access to this ATCC deposit will be available during the pendency of the application to the Commissioner of Patents and Trademarks and persons determined by the Commissioner to be entitled thereto upon request, in accordance with applicable laws and regulations. Upon issuance of a patent, this deposit of seed of maize Event DP-202216-6 is intended to meet all the necessary requirements of 37 C.F.R. §§ 1.801-1.809, and will be maintained in the ATCC depository, for a period of 30 years, or 5 years after the most recent request, or for the enforceable life of the patent, whichever is longer, and will be replaced if it becomes nonviable during that period. Unauthorized seed multiplication prohibited. The seed may be regulated under one or more applicable National, State or other local regulations and ordinances imposed by one or more competent governmental agencies.

As used herein, the term “corn” means Zea mays or maize and includes all plant varieties that can be bred with corn, including wild maize species.

As used herein, the terms “insect resistant” and “impacting insect pests” refers to effecting changes in insect feeding, growth, and/or behavior at any stage of development, including but not limited to: killing the insect; retarding growth; reducing reproductive capability; inhibiting feeding; and the like.

As used herein, the terms “pesticidal activity” and “insecticidal activity” are used synonymously to refer to activity of an organism or a substance (such as, for example, a protein) that can be measured by numerous parameters including, but not limited to, pest mortality, pest weight loss, pest attraction, pest repellency, and other behavioral and physical changes of a pest after feeding on and/or exposure to the organism or substance for an appropriate length of time. For example, “pesticidal proteins” are proteins that display pesticidal activity by themselves or in combination with other proteins.

As used herein, “insert DNA” refers to the heterologous DNA within the expression cassettes used to transform the plant material while “flanking DNA” can exist of either genomic DNA naturally present in an organism such as a plant, or foreign (heterologous) DNA introduced via the transformation process which is extraneous to the original insert DNA molecule, e.g. fragments associated with the transformation event. A “flanking region” or “flanking sequence” as used herein refers to a sequence of at least 20 bp, for some embodiments, at least 50 bp, and up to 5000 bp, which is located either immediately upstream of and contiguous with or immediately downstream of and contiguous with the original foreign insert DNA molecule.

In an embodiment, the junction sequences of Event DP-202216-6, for example, may include polymorphisms (e.g., SNPs) or mutations that may occur spontaneously in the endogenous genomic region of the junction sequence. These may include insertion, deletion or substitution of one or more nucleotides in the junction sequence. Polynucleotide sequences that are at least 90%, 91%, 92%, 93%, 94%, 95%, 96%, 97%, 98%, and 99% to one or more of the junction sequences.

As used herein, “heterologous” in reference to a nucleic acid sequence is a nucleic acid sequence that originates from a foreign species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

The term “regulatory element” refers to a nucleic acid molecule having gene regulatory activity, i.e. one that has the ability to affect the transcriptional and/or translational expression pattern of an operably linked transcribable polynucleotide. The term “gene regulatory activity” thus refers to the ability to affect the expression of an operably linked transcribable polynucleotide molecule by affecting the transcription and/or translation of that operably linked transcribable polynucleotide molecule. Gene regulatory activity may be positive and/or negative and the effect may be characterized by its temporal, spatial, developmental, tissue, environmental, physiological, pathological, cell cycle, and/or chemically responsive qualities as well as by quantitative or qualitative indications.

“Promoter” refers to a nucleotide sequence capable of controlling the expression of a coding sequence or functional RNA. In general, a coding sequence is located 3′ to a promoter sequence. The promoter sequence comprises proximal and more distal upstream elements, the latter elements are often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different regulatory elements may direct the expression of a gene in different tissues or cell types, or at different stages of development, or in response to different environmental conditions. Promoters that cause a nucleic acid fragment to be expressed in most cell types at most times are commonly referred to as “constitutive promoters”.

A DNA construct is an assembly of DNA molecules linked together that provide one or more expression cassettes. The DNA construct may be a plasmid that is enabled for self-replication in a bacterial cell and contains various endonuclease enzyme restriction sites that are useful for introducing DNA molecules that provide functional genetic elements, i.e., promoters, introns, leaders, coding sequences, 3′ termination regions, among others; or a DNA construct may be a linear assembly of DNA molecules, such as an expression cassette. The expression cassette contained within a DNA construct comprises the necessary genetic elements to provide transcription of a messenger RNA. The expression cassette can be designed to express in prokaryote cells or eukaryotic cells. Expression cassettes of the embodiments are designed to express in plant cells.

The DNA molecules disclosed herein are provided in expression cassettes for expression in an organism of interest. The cassette will include 5′ and 3′ regulatory sequences operably linked to a coding sequence. “Operably linked” means that the nucleic acid sequences being linked are contiguous and, where necessary to join two protein coding regions, contiguous and in the same reading frame. Operably linked is intended to indicate a functional linkage between a promoter and a second sequence, wherein the promoter sequence initiates and mediates transcription of the DNA sequence corresponding to the second sequence. The cassette may additionally contain at least one additional gene to be co-transformed into the organism. Alternatively, the additional gene(s) can be provided on multiple expression cassettes or multiple DNA constructs.

The expression cassette may include in the 5′ to 3′ direction of transcription: a transcriptional and translational initiation region, a coding region, and a transcriptional and translational termination region functional in the organism serving as a host. The transcriptional initiation region (i.e., the promoter) may be native or analogous, or foreign or heterologous to the host organism. Additionally, the promoter may be the natural sequence or alternatively a synthetic sequence. The expression cassettes may additionally contain 5′ leader sequences in the expression cassette construct. Such leader sequences can act to enhance translation.

A transgenic “event” is produced by transformation of plant cells with a heterologous DNA construct(s), including a nucleic acid expression cassette that comprises a transgene of interest, the regeneration of a population of plants resulting from the insertion of the transgene into the genome of the plant, and selection of a particular plant characterized by insertion into a particular genome location. An event is characterized phenotypically by the expression of the transgene. At the genetic level, an event is part of the genetic makeup of a plant. The term “event” also refers to progeny produced by a sexual outcross between the transformant and another variety that include the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

Corn plant containing event DP-202216-6 may be bred by first sexually crossing a first parental corn plant consisting of a corn plant grown from event DP-202216-6 corn plant and progeny thereof derived from transformation with the expression cassettes of the embodiments that increase yield when compared to a control plant, and a second parental corn plant that does not have such constructs, thereby producing a plurality of first progeny plants; and then selecting a first progeny plant that demonstrates yield increase; and selfing the first progeny plant, thereby producing a plurality of second progeny plants; and then selecting from the second progeny plants plant with yield increase.

As used herein, the term “plant” includes reference to whole plants, parts of plants, plant organs (e.g., leaves, stems, roots, etc.), seeds, plant cells, and progeny of same. In some embodiments, parts of transgenic plants comprise, for example, plant cells, protoplasts, tissues, callus, embryos as well as flowers, stems, fruits, leaves, and roots originating in transgenic plants or their progeny previously transformed with a DNA molecule disclosed herein, and therefore consisting at least in part of transgenic cells.

As used herein, the term “plant cell” includes, without limitation, seeds, suspension cultures, embryos, meristematic regions, callus tissue, leaves, roots, shoots, gametophytes, sporophytes, pollen, and microspores. The class of plants that may be used is generally as broad as the class of higher plants amenable to transformation techniques, including both monocotyledonous and dicotyledonous plants.

The present disclosure provides a commodity product that is derived from a corn plant comprising event DP-202216-6. As used herein, a “commodity product” generally refers to any composition or material that includes material derived or processed from a plant, seed, plant cell, or plant part comprising event DP-202216-6. Commodity products may be viable (e.g., seeds) or nonviable (e.g., corn meal). Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal's consumption, oil, meal, flour, flakes, bran, fiber, milk, cheese, paper, cream, wine, ethanol, and any other food for human consumption; and biomasses and fuel products. Viable commodity products include but are not limited to seeds and plant cells.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050, incorporated herein by reference). Additional transformation methods are disclosed below.

As used herein, the term “progeny” in the context of DP-202216-6 denotes the offspring of any generation of a parent plant which comprises corn event DP-202216-6.

Isolated polynucleotides disclosed herein may be incorporated into recombinant constructs, typically DNA constructs, which are capable of introduction into and replication in a host cell. Such a construct may be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., (1985; Supp. 1987) Cloning Vectors: A Laboratory Manual, Weissbach and Weissbach (1989) Methods for Plant Molecular Biology, (Academic Press, New York); and Flevin et al., (1990) Plant Molecular Biology Manual, (Kluwer Academic Publishers). Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

During the process of introducing an insert into the genome of plant cells, it is not uncommon for some deletions or other alterations of the insert and/or genomic flanking sequences to occur. Thus, the relevant segment of the plasmid sequence provided herein might comprise some minor variations. The same is true for the flanking sequences provided herein. Thus, a plant comprising a polynucleotide having some range of identity with the subject flanking and/or insert sequences is within the scope of the subject disclosure. Identity to the sequence of the present disclosure may be a polynucleotide sequence having at least 65% sequence identity, for some embodiments at least 70% sequence identity, for some embodiments at least 75% sequence identity, for some embodiments at least 80% identity, and for some embodiments at least 85% 86%, 87%, 88%, 89%, 90%, 91%, 92%, 93%, 94%, 95%, 96%, 97%, 98%, 99%, 99.5% sequence identity with a sequence exemplified or described herein. Hybridization and hybridization conditions as provided herein can also be used to define such plants and polynucleotide sequences of the subject disclosure. The sequence which comprises the flanking sequences plus the full insert sequence can be confirmed with reference to the deposited seed.

A “probe” is an isolated nucleic acid to which is attached a conventional detectable label or reporter molecule, e.g., a radioactive isotope, ligand, chemiluminescent agent, or enzyme. Such a probe is complementary to a strand of a target nucleic acid, for example, to a strand of isolated DNA from corn event DP-202216-6 whether from a corn plant or from a sample that includes DNA from the event. Probes may include not only deoxyribonucleic or ribonucleic acids but also polyamides and other probe materials that bind specifically to a target DNA sequence and can be used to detect the presence of that target DNA sequence.

“Primers” are isolated nucleic acids that anneal to a complementary target DNA strand by nucleic acid hybridization to form a hybrid between the primer and the target DNA strand, then extended along the target DNA strand by a polymerase, e.g., a DNA polymerase. Primer pairs refer to their use for amplification of a target nucleic acid sequence, e.g., by PCR or other conventional nucleic-acid amplification methods. “PCR” or “polymerase chain reaction” is a technique used for the amplification of specific DNA segments (see, U.S. Pat. Nos. 4,683,195 and 4,800,159; herein incorporated by reference).

A nucleic acid molecule is said to be the “complement” of another nucleic acid molecule if they exhibit complete complementarity or minimal complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, a Practical Approach, IRL Press, Washington, D.C. (1985), departures from complete complementarity are therefore permissible, as long as such departures do not completely preclude the capacity of the molecules to form a double-stranded structure. In order for a nucleic acid molecule to serve as a primer or probe, it needs to be sufficiently complementary in sequence to be able to form a stable double-stranded structure under the particular solvent and salt concentrations employed.

In hybridization reactions, specificity is typically the function of post-hybridization washes, the relevant factors being the ionic strength and temperature of the final wash solution. The thermal melting point (Tₘ) is the temperature (under defined ionic strength and pH) at which 50% of a complementary target sequence hybridizes to a perfectly matched probe. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° ° C. lower than the Tₘ.

Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C.(aqueous solution) or 32° C.(formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) and Sambrook et al. (1989).

The principle of hybridization analysis is that a single-stranded DNA or RNA molecule of a known sequence (e.g., the probe) can base-pair to a second DNA or RNA molecule that contains a complementary sequence (the target), with the stability of the hybridization depending on the extent of base pairing that occurs under the conditions tested. Appropriate stringency conditions for DNA hybridization, include for example, 6×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. or up to 0.1×SSC or 0.2×SSC, at 55° C. or 65° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable (e.g., time) is changed. In one embodiment, a nucleic acid of the present disclosure will specifically hybridize to one or more of the nucleic acid molecules or complements or fragments thereof under high stringency conditions. The hybridization of the probe to the target DNA molecule can be detected by methods known to those skilled in the art. These can include, but are not limited to, fluorescent tags, radioactive tags, antibody-based tags, and chemiluminescent tags.

In some embodiments, a complementary sequence has the same length as the nucleic acid molecule to which it hybridizes. In some embodiments, the complementary sequence is 1, 2, 3, 4, 5, 6, 7, 8, 9, or 10 nucleotides longer or shorter than the nucleic acid molecule to which it hybridizes. In some embodiments, the complementary sequence is 1%, 2%, 3%, 4%, or 5% longer or shorter than the nucleic acid molecule to which it hybridizes. In some embodiments, a complementary sequence is complementary on a nucleotide-for-nucleotide basis, meaning that there are no mismatched nucleotides (each A pairs with a T and each G pairs with a C). In some embodiments, a complementary sequence comprises 1, 2, 3, 4, 5, 6, 7, 8, 9, 10 or less mismatches. In some embodiments, the complementary sequence comprises 1%, 2%, 3%, 4%, 5%, 6%, 7%, 8%, 9%, or 10% or less mismatches.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100). For example, Clustal W method of aligning multiple sequences is described in Thompson J, Higgins D and Gibson T (1994). Clustal W: improving the sensitivity of progressive multiple sequence alignment through sequence weighting.” Nucleic Acids Research, Vol 22: pp. 4673-80. Another method is Clustal V, described in Higgins DG and Sharp PM (1989). “Fast and sensitive multiple sequence alignments on a microcomputer.” CABIOS, Vol. 5, No. 2: pp. 151-153.

Regarding the amplification of a target nucleic acid sequence (e.g., by PCR) using a particular amplification primer pair, stringent conditions permit the primer pair to hybridize only to the target nucleic-acid sequence to which a primer having the corresponding wild-type sequence (or its complement) would bind to produce a unique amplification product, the amplicon, in a DNA thermal amplification reaction.

The term “allele” refers to an alternative form of a gene, whereby two genes can differ in DNA sequences. Such differences may result from at least one mutation (e.g., deletion, insertion, and/or substitution) in the nucleic acid sequence. Alleles may result in modified mRNAs or polypeptides whose structure or function may or may not be modified. Any given gene may have none, one, or many allelic forms. Each of these types of changes may occur alone, or in combination with the others, one or more times in a given sequence.

A hybridization reaction using a probe specific to a sequence found within the amplicon is yet another method used to detect the amplicon produced by a PCR reaction. The term “zygosity” generally refers to the similarity of alleles for a gene or trait in an organism (e.g., a plant). If both alleles are the same, the organism is homozygous for the allele. If the two alleles are different, the organism is heterozygous for the gene or trait. If one allele is not present, the organism is hemizygous. If both alleles are not present, the organism is nullizygous. For example, a plant is homozygous for the trait of interest if the insert DNA along with the junction sequence is present at the same location on each chromosome of a chromosome pair (both the alleles). For example, a maize plant having Event DP-202216-6 at the same location on both the copies of the chromosome. Similarly, a plant is considered heterozygous if the transgene insert along with the junction sequence (e.g., Event DP-202216-6) is present on only one of the chromosomes of a chromosome pair (only one allele). A wild-type plant is considered “null” when compared to the transgenic Event DNA.

As used herein, a “line” is a group of plants that display little or no genetic variation between individuals for at least one trait. Such lines may be created by several generations of self-pollination and selection, or vegetative propagation from a single parent using tissue or cell culture techniques.

As used herein, the terms “cultivar” and “variety” are synonymous and refer to a line which is used for commercial production. “Stability” or “stable” means that with respect to the given component, the component is maintained from generation to generation and, for some embodiments, at least three generations at substantially the same level, e.g., for some embodiments±15%, for some embodiments±10%, most for some embodiments±5%. The stability may be affected by temperature, location, stress and the time of planting.

“Agronomically elite” means that a line has desirable agronomic characteristics such as maturity, disease resistance, standability, ear height, plant height, and the like, in addition to yield increase due to the subject event(s).

As used herein, the term “stacked” or “stacked traits” refers to having multiple traits present in the same plant or organism of interest. A trait, as used herein, refers to the phenotype derived from a particular sequence or groups of sequences. In one embodiment, the stacked traits comprise at least one stress tolerant trait.

In some embodiments the maize plants disclosed herein having yield trait (e.g., increased and extended expression of AG099) may further comprise a stack of additional traits. Plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). Additional traits can include for example, drought tolerance and other abiotic stress tolerance traits. Such traits can be introduced by breeding with maize plants containing other recombinant events or with maize plants containing native variations or genome edited variations.

In some embodiments the maize plants disclosed herein having yield trait (e.g., increased and extended expression of AG099) can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). In a further embodiment, the DP-202216-6 maize event may be combined with one or more additional Bt insecticidal toxins or other non-Bt insecticidal proteins.

In an aspect, a corn field has plants that have the MADS-box transcription factor disclosed herein along with introduced genetic modifications affecting stature, and includes a planting density of at least 10,000 corn plants per acre. In another aspect, a corn field comprises a planting density of at least 30,000 corn plants per acre. In another aspect, a corn field includes a planting density of at least 32,000 corn plants per acre. In another aspect, a corn field includes a planting density of at least 34,000 corn plants per acre. In another aspect, a corn field includes a planting density of at least 36,000 corn plants per acre. In another aspect, a corn field includes a planting density of at least 38,000 corn plants per acre. In another aspect, a corn field includes a planting density of at least 40,000 corn plants per acre. In another aspect, a corn field includes a planting density of at least 42,000 corn plants per acre. In another aspect, a corn field includes a planting density of at least 44,000 corn plants per acre. In another aspect, a corn field includes a planting density of at least 46,000 corn plants per acre. In another aspect, a corn field includes a planting density of at least 48,000 corn plants per acre. In another aspect, a corn field includes a planting density of at least 50,000 corn plants per acre. In another aspect, a corn field includes a planting density of at least 52,000 corn plants per acre. In another aspect, a corn field includes a planting density of at least 54,000 corn plants per acre. In another aspect, a corn field includes a planting density of at least 56,000 corn plants per acre. In another aspect, a corn field includes a planting density of at least 58,000 corn plants per acre. In another aspect, a corn field includes a planting density of at least 60,000 corn plants per acre.

In an aspect, a corn field has plants that have the MADS-box transcription factor, the corn plants containing MADS-box transcription factor expressing at a higher level in combination with shorter stature or semi-dwarf phenotype exhibits plant that is at least about 10%, between 10% and 15%, at least about 15%, between 15% and 20%, at least about 20%, between 20% and 25%, at least about 25%, between 25% and 30%, at least about 30%, between 30% and 35%, at least about 35%, between 35% and 40%, at least about 40%, between 40% and 45%, at least about 45%, between 45% and 50%, at least about 50%, between 50% and 55%, at least about 55%, between 55% and 60%, at least about 60%, between 60% and 65%, at least about 65%, between 65% and 70%, at least about 70%, between 70% and 75%, at least about 75%, between 75% and 80%, when compared to the plant height of control corn plants not comprising the stature genetic modification, when measured at or near reproductive/late reproductive growth phase (e.g., R1-R6).

In an aspect, a corn field has plants that have the MADS-box transcription factor disclosed herein along with introduced genetic modifications affecting stature and includes a planting density of between 10,000 and 50,000 corn plants per acre. In an aspect, a corn field includes a planting density of between 10,000 and 40,000 corn plants per acre. In an aspect, a corn field includes a planting density of between 10,000 and 30,000 corn plants per acre. In an aspect, a corn field includes a planting density of between 10,000 and 25,000 corn plants per acre. In an aspect, a corn field includes a planting density of between 10,000 and 20,000 corn plants per acre. In an aspect, a corn field includes a planting density of between 20,000 corn plants and 60,000 corn plants per acre. In an aspect, a corn field includes a planting density of between 20,000 corn plants and 58,000 corn plants per acre. In an aspect, a corn field includes a planting density of between 20,000 corn plants and 55,000 corn plants per acre. In an aspect, a corn field includes a planting density of between 20,000 corn plants and 50,000 corn plants per acre. In an aspect, a corn field includes a planting density of between 20,000 corn plants and 45,000 corn plants per acre. In an aspect, a corn field includes a planting density of between 20,000 corn plants and 42,000 corn plants per acre. In an aspect, a corn field includes a planting density of between 20,000 corn plants and 40,000 corn plants per acre. In an aspect, a corn field includes a planting density of between 20,000 corn plants and 38,000 corn plants per acre. In an aspect, a corn field includes a planting density of between 20,000 corn plants and 36,000 corn plants per acre. In an aspect, a corn field includes a planting density of between 20,000 corn plants and 34,000 corn plants per acre. In an aspect, a corn field includes a planting density of between 20,000 corn plants and 32,000 corn plants per acre. In an aspect, a corn field includes a planting density of between 20,000 corn plants and 30,000 corn plants per acre. In an aspect, a corn field includes a planting density of between 24,000 corn plants and 58,000 corn plants per acre. In an aspect, a corn field includes a planting density of between 38,000 corn plants and 60,000 corn plants per acre. In an aspect, a corn field includes a planting density of between 38,000 corn plants and 50,000 corn plants per acre.

In some embodiments the maize plants disclosed herein having yield trait (e.g., increased and extended expression of AG099) can be crossed with corn plants containing other corn Events or combination thereof and the resulting properties of the progeny plants are evaluated. For example, corn plants containing DP-202216-6 Event can be crossed or combined with corn plants including one or more combinations, of the following: MON810; DAS-59122-7; MIR604; MON89034; MON863; MON87411; MON87403; MON87427; MON-00603-6 (NK603); MON-87460-4; MON-88017-3; LY038; TC1507; 5307; DAS-06275-8; BT176; BT11; MIR162; GA21; MZDT09Y; SYN-05307-1; DP-004114-3; and DAS-40278-9.

The following examples are offered by way of illustration and not by way of limitation. As described herein, Event DP-202216-6 is also referred to as “Event 16”, “E16” “event 16” or “Event 16-6” and they all refer to the same maize event DP-202216-6. The protein encoded by the Maize MADS box ZmM28 gene in the plasmid PHP40099 or the Event DP-202216-6 is also referred to as AG099 protein and the corresponding DNA sequence as AG099 gene or AG099 DNA.

### EXAMPLES

### Example 1

### Field Performance of Maize Plants Having Increased and Extended Expression of AG099 at Various Planting Densities and Applied Nitrogen Fertilizer

Transformed maize plants containing the AG099 trait are converted into elite inbreds and are top-crossed for a series of grain yield trials. The efficacy and improvement of the AG099 trait towards maize hybrid yield is enhanced when delivered from the female side of a hybrid cross.

A series of trials are conducted in elite corn hybrids across testing locations in order to assess the field performance of maize plants (e.g., maize hybrids) and having AG099 events. For example, hybrids with maturities ranging from 105-112 days are used to evaluate the performance of AG099 events relative to controls.

In certain aspects, locations selected to achieve various yield levels can range from highly drought stressed 70 bu/acre to optimal growing conditions 250 bu/acre. Soil types include a variety of high sand, sandy loam, silty loam, loam and some clay. In such locations, entries containing the AG099 construct are compared to its control for each specific hybrid background. Two to four replicates of a split plot design are established at each location. A mixed model analysis of variance can be conducted using ASREML where BLUEs (Best Linear Unbiased Estimates) are generated for each AG099 event in combination. Pairwise contrasts of these event BLUEs to wild type BLUEs are analyzed to test significant differences.

In an experimental location, 4 replicates of 4 hybrids (2 hybrid platforms×2 events) with controls were tested at 5 different planting densities with 2 different Nitrogen treatments as follows (Table 1):

Nitrogen Treatments

| Entry Name | Pre-Plant (lbs. N) | V4-V5 (lbs. N) | Total Kg/ha |
|---|---|---|---|
| 40# | 40 | 0 | 44.8 |
| 240# | 40 | 200 | 268.8 |

The planting densities ranged from 20,000 to 44,000 plants per acre.

In another experiment, various applied nitrogen levels were evaluated at different planting densities (34,000 and 44,000 plants per acre) as follows (Table 2).

Nitrogen Treatments

| Entry Name | Pre-Plant (lbs. N) | V4-V5 (lbs. N) | Total Kg/ha |
|---|---|---|---|
| 0# | 0 | 0 | 0 |
| 50 PRE | 50 | 0 | 56 |
| 100 Split | 50 | 50 | 112.1 |
| 200 SD | 0 | 200 | 224.2 |
| 200 Split | 50 | 150 | 224.2 |

In another experiment, reduced N rate was evaluated at 5 different planting densities (20000; 26000; 32000; 38000 and 44000), 4 replicates, two N treatments and 4 hybrids as follows (Table 3):

Nitrogen Treatments

| Name | Pre-Plant (lbs. N) | V4-V5 (lbs. N) | Total Kg/ha |
|---|---|---|---|
| 80% | 0 | 175 | 196 |
| 100 | 0 | 220 | 246 |

In certain aspects, such as for example, high-yielding where yields of over 160 bu/acre are considered as normal and often can be greater than 180 bu/acre which represents a large portion of the most productive corn growing regions in the United States. In order to evaluate the response of corn hybrids containing AG099 event across a matrix of planting densities and N treatments provide a prescription of hybrids, N application rates and planting densities to maximize sustainability.

### Example 2

### Field Performance of Maize Plants Having Increased and Extended Expression of AG099 Under Nitrogen Stabilization Conditions

To test AG099 containing events under applied N stabilizer, experiments are conducted at multiple locations and at varying planting population or planting density at a variety of applied N and applied Nitrogen stabilizers along with such applied N.

Partial factor productivity (PFP) of N (also referred to as PFPN) is a measure of how many pounds of grain are produced per pound of applied fertilizer N. This productivity factor is also a measure of N use efficiency (NUE) at the field level. Depending on the N measurement, PFP or PFPN is a measure N productivity in a field, not taking into account the different types of N sources (e.g., fertilizer N, soil organic matter, mineralization, residual nitrate, manure and fixed nitrogen from crop rotations). Nevertheless, when the loss of N is high (e.g., volatile N, N runoffs) PFPN will decrease. Lower PFPN is a sign of lower grain productivity per applied N or grain parity but at a significantly higher applied N. Loss of N depends on fertilizer management practices such as N application timing, N source, N placement, and N rate, and environmental conditions such soil type, topography and weather. To reduce the loss of N due to environmental conditions, N fertilizer stabilizer is used. Nitrogen stabilizers can be nitrification inhibitors, urease inhibitors, or a combination thereof.

Crops use nitrogen in two forms: ammonium (NH4+) and nitrate (NO3−). Plants prefer ammonium form since that N form is easier to absorb and less susceptible to loss. Nitrification inhibitors (NIs) are often mixed with ammonium-forming N fertilizers to decrease the rate of conversion of ammonium (NH4+) to nitrate (NO3−). The nitrate form is prone to leaching and denitrification and often accounts for substantial loss of applied N (up to or greater than 50% of applied fertilizer N applied on sandy soils). The magnitude of positive impact on grain yield because of the use of NIs depends on the environmental conditions (e.g., heavy rainfall after N application) and soil type (e.g., sandy). Urease inhibitors (UI) are often mixed with urea-based fertilizers to decrease the rate of urea hydrolysis by reducing or inhibiting the activity of urease enzyme. UIs are more effective when applied with based fertilizers (surface-applied) where soil pH is high and soil residue is high (e.g., due to no-till or low till) which contribute to high residual urease activity in soil. Urea hydrolysis can often lead to about 30% fertilizer N lost through ammonia volatilization.

Several compounds are effective for stabilizing nitrogen, including nitrapyrin, dicyandiamide, and ammonium thiosulfate. Nitrapyrin, or 2-chloro-6-(trichloromethyl) pyridine, works by inhibiting or reducting the activity of Nitrosomonas bacteria. Nitrapyrin has a bactericidal effect, inhibiting the growth of Nitrosomonas population in the soil. Field performance of maize plants expressing a transgenic yield trait (e.g., increased and extended expression of AG099) in the presence of one or more nitrogen stabilizers is evaluated. Label rates of nitrogen stabilizers is used along with variations in the amount that ranged from 10% to about 100% of the nitrogen stabilizer application rates. A range of application timings are also evaluated, which includes pre-plant, early season and late season N application.

Applied nitrogen is optimized in a field by a population of crop plants that contain a transgenic trait affecting yield, the method includes providing crop plants wherein the expression of a transgenic trait increases one or more yield related agronomic parameters to a field that comprises applied nitrogen fertilizer and a nitrogen stabilizer; and increasing the nitrogen utilization rate during the crop plants growing season compared to a control population of plants not comprising the trait. In this Example, the plants are corn plants that comprise a heterologous polynucleotide that encodes a MADS-box polypeptide. More specifically, the corn plants are planted in one or more zones within the field, where the zones are characterized by nitrogen run-offs. Further, in an experiment, the applied nitrogen is about 10% to about 50% less per acre compared to a normal field not comprising the crop plants having the transgenic trait, in the presence of a nitrogen stabilizer. Maize plants expressing AG099 are more effective in utilizing N through its various development cycle to maximize grain yield per applied N. In the presence of N stabilizer, the N utilization efficiency for AG099 expressing plants is higher compared to control plants not expressing AG099 at increased and extended level. Thus, the applied nitrogen is used more effectively by the crop plants as measured by increase in stalk and/or leaf nitrogen content; the applied nitrogen is used more effectively by the crop plants as measured by increase in root length and/or root density compared to a control population of plants not comprising the trait; the applied nitrogen is used more effectively by the crop plants as measured by increase in shoot and/or root biomass. In certain embodiments, the plants are corn plants that comprise event DP-202216-6. Applied nitrogen is about 5% to about 50% less than an application rate of about 50 lbs to about 400 lbs of nitrogen per acre compared to a normal field not comprising the crop plants having the transgenic trait.

### Example 3

Crop Rotation, Sustainable Agronomic Practices with Maize Plants Containing Increased Expression of AG099

Crop rotation methods directed to maize plants expressing a transgenic yield trait (e.g., increased and extended expression of AG099) are provided herein. Rotating crops is one of the practices recommended for high yields of corn. However, the field performance of the second crop following maize plants expressing a transgenic yield trait (e.g., increased and extended expression of AG099) has not been established. Crop rotation can break damaging insect and disease cycles that lower crop yields. Including crops such as soybean or alfalfa in the rotation can reduce the amount of nitrogen required in the following corn crop; however, the performance of maize plants expressing a transgenic yield trait (e.g., increased and extended expression of AG099) has not been shown.

In a majority of fields with high yielding corn acres (e.g., 250-300 bu/acre), corn is planted following a crop other than corn in the previous growing season. This “rotation effect” refers to a yield increase associated with crop rotation compared to continuous corn growing seasons when the generally associated limiting factors have been controlled or adequately supplied/addressed in the continuous corn. This yield increase has averaged about 5% to 15% (compared to controls) but has generally been less under high-yield conditions. Field performance of maize plants expressing a transgenic yield trait (e.g., increased and extended expression of AG099) under continuous corn and crop rotation farming practices are evaluated. Rotated corn is generally better able to tolerate yield-limiting stresses than continuous corn, but with sustainable agricultural practices, performance of maize plants expressing a transgenic yield trait (e.g., increased and extended expression of AG099) is evaluated where the amount of nitrogen supplied per acre is controlled to measure the effects of crop rotation's effect on corn yield as well as the second crop's performance (e.g., soybean, cotton or alfalfa).

Tilling practices involving maize plants expressing a transgenic yield trait (e.g., increased and extended expression of AG099) are evaluated. For example, field performance of these plants with conventional tillage, no-tillage or some type of reduced tillage (e.g., strip, minimum, ridge, mulch) are evaluated. These AG099 expressing maize plants perform better despite factors such as hairpinning, sidewall compaction, lack of consistent seeding depth, and failure of the furrow to close properly over the seed, reduction in seed-to-soil contact and show improved stand establishment in high-residue systems.

On an average, corn grain removes approximately 0.67 lbs of N per bushel harvested, and stover production requires about 0.45 lbs of N for each bushel of grain produced. This utilization factor equates, as measure of total N needed for a 300 bu/acre corn crop is about 336 lbs/acre. However, not all of this N is supplied as exogenously applied fertilizer. Nis also supplied by the soil through mineralization of soil organic matter, especially on highly productive soils, N mineralization can supply a significant portion of N needed by the corn crop. N can also obtained from N fixed by the previous legume crop, manure application, and N in irrigation water. The maize plants expressing a transgenic yield trait (e.g., increased and extended expression of AG099) are expected to yield better when compared to control plants under these applied N conditions.

Timing of N fertilizer applications is also a factor in addition to the application rate. Reduction in time between N application and crop uptake results in lower N loss from the soil and enhances crop yield. Maize plants expressing a transgenic yield trait (e.g., increased and extended expression of AG099) with improved early germination and vigor, less N is lost and increases the overall yield. Stabilized N, either in the form of delayed release fertilizers or the application of nitrogen stabilizers such as nitrapyrin, increase the availability of N especially beginning at V6 and extending to the R5 (early dent) stage of grain development. Recommended timing of N fertilizer include at planting or pre-plant. Fall-applied N may not be needed. Some form of in-season N, either side-dressed or applied with irrigation is also recommended to increase crop yields of maize plants expressing a transgenic yield trait (e.g., increased and extended expression of AG099).

Micronutrients such as sulfur (S) and zinc (Zn) are also applied with varying amounts depending on soil type for fields containing maize plants expressing a transgenic yield trait (e.g., increased and extended expression of AG099). In certain soil types, this micronutrient application also includes boron (B), magnesium (Mg), manganese (Mn), or copper (Cu). Because maize plants expressing a transgenic yield trait (e.g., increased and extended expression of AG099) exhibit increased yield, these micronutrients in soil may be utilized to a greater extent requiring additional replenishment for continued high yield performance.

Methods of rotating crops in one or more consecutive growing seasons includes growing a population of transgenic corn plants in a first growing season in a field, wherein the expression of a polynucleotide that encodes a MADS-box polypeptide is increased due to a genetic modification, compared to a control plant; growing a population of a second crop plants in a second growing season in the field, wherein the second growing season is consecutive to the first growing season and wherein the second crop plant is not corn; and increasing yield of the population of the soybean plants compared to a control population of soybean plants grown in a control field. In certain embodiments, corn plants comprise event DP-202216-6. In certain planting cycles, the population of second crop plants is selected from the group consisting of soybeans, cotton, wheat and sorghum.

Maize plants expressing a transgenic yield trait (e.g., increased and extended expression of AG099) increase yield of the second or a subsequent crop due to increased sequestration of soil organic matter and/or nitrogen. The yield increase of the second crop is improved when the corn plants have early maturity rating and are planted early during the first growing season such that the population of the second crop plants are planted early in the second growing season, the first and second growing seasons are complete within about 8-12 months. Before planting the maize plants expressing a transgenic yield trait (e.g., increased and extended expression of AG099 or a MADS-box polypeptide), the field is not tilled, practice conservation tilling, minimum tilling or strip till. In certain planting cycles, the corn plants are selected to be in a relative maturity zone 80 CRM to about 120 CRM and wherein the second crop plants are soybean plants selected to be in a relative maturity zone of Group 2 to Group 6.

### Example 4

### AG099 Plants, Short Stature AG099 Plants and Cover Crop Agronomic Practices

Adding cover crops to corn and soybean cropping systems improves sustainability practices. Cover crops offer opportunities for improving soil quality and crop production efficiency. Cover crops provide a long-term investment in soil productivity. For example, legume cover crops can fix valuable nitrogen (N) for a subsequent corn crop. Legumes take longer to establish than grasses in the fall and are less well suited for scavenging nutrients, suppressing weeds and protecting the soil from erosion. Selecting the right cover crop for maize plants expressing a transgenic yield trait (e.g., increased and extended expression of AG099 or a MADS-box polypeptide) increases the likelihood to maximize yield and provide sustainable benefits to the environment.

The most commonly used cover crops fall into 1 of 3 broad groups based on species, potential benefits and management considerations. Grasses, including winter cereals such as rye, wheat, barley and triticale, are widely used cover crops in corn and soybean cropping systems. Winter cereals are typically planted in late summer through late fall and produce a small to moderate amount of root and above-ground biomass before going dormant in the winter. Non-winter-hardy cereals like oats, which establish rapidly in the fall, are also suitable and they leave behind little residue to manage in the spring. Annual ryegrass is another option if spring residue levels are a concern. Annual ryegrass is a bunch-type forage grass that produces less above-ground biomass than winter cereals but more root biomass. Annual ryegrass is slower to establish in the fall compared to winter cereals and are seeded by mid-September/early Fall in most locations to survive the winter. Because it produces a large amount of shallow root biomass, annual ryegrass is a good fit for no-till systems.

Legumes are valued as cover crops primarily for their ability to fix N. Common legumes used as winter cover crops in corn and soybean cropping systems include hairy vetch, field pea, lentil, crimson clover, red clover and berseem clover. Legumes can be seeded in early summer through early fall but in many regions must be planted earlier than cereals to survive the winter. Brassica cover crops provide similar benefits as grasses and their residues break down more rapidly in the Spring. Certain brassicas produce a large taproot that is effective at breaking soil compaction. Common brassicas used as winter cover crops in corn and soybean cropping systems include canola, mustards, forage radish and turnip.

In field that contain maize plants expressing a transgenic yield trait (e.g., increased and extended expression of AG099 or a MADS-box polypeptide), mixtures of 2 or more cover crops are often superior to a single species (“pure stands”). Grass-legume mixtures can be particularly advantageous because they combine the benefits of both—quick soil cover and N scavenging by grasses and N additions by legumes. The presence of N-rich legume residues can also help break down grass residues more quickly in the spring. Cover crop establishment is managed by seeding date, soil type, soil moisture, existing crop's ground cover, and other agronomic practices.

Seeding dates of cover crops for maize plants expressing a transgenic yield trait (e.g., increased and extended expression of AG099 or a MADS-box polypeptide) and for those maize plants that are short stature, include in-season seeding into standing corn can be an effective establishment method for certain cover crops in short-season environments. In general, legumes, brassicas and other small-seeded species with extended seedling growth are candidates for in-season seeding. Planting cereals and other large-seeded cover crops far in advance of corn is not recommended, as these species have a higher initial light requirement. If seeding occurs prior to layby, cover crops can be planted with an N side-dress applicator or by attaching seed boxes to a row cultivator. High-clearance or aerial seeding equipment is required if cover crop seeding takes place after canopy closure or when there is early canopy closure due to high-density planting and/or short stature AG099 plants or short stature corn plants.

Seeding at physiological maturity of maize plants expressing a transgenic yield trait (e.g., increased and extended expression of AG099 or a MADS-box polypeptide) can provide additional time for establishing grasses and grass-legume mixtures in central and northern locations of the continental US and Canada. As the grain crop dries down, sunlight breaks through the canopy and improves conditions for germination and early cover crop growth. Seeding cover crops into mature corn and soybeans may need high-clearance or aerial seeding equipment for normal height corn. Postharvest seeding after maize plants expressing a transgenic yield trait (e.g., increased and extended expression of AG099 or a MADS-box polypeptide) are harvested is also an option. However, this can restrict cover crop options in some years and locations, and reduce establishment success. In areas with a longer growing season or in cases where corn is harvested for silage, most grass and legume cover crops can be successfully planted following crop harvest. In northern locations, winter small grains are best suited for postharvest seeding. Grain drills, row crop planters, broadcast seeders and fertilizer floaters can all be used to seed cover crops following grain crop harvest.

For maize plants expressing a transgenic yield trait (e.g., increased and extended expression of AG099 or a MADS-box polypeptide), numerous methods can be used to seed winter cover crops. These methods depend largely on the time of seeding, but cover crop species and farming operation considerations are also factors. Common methods for seeding winter cover crops include grain drills (a reliable method for seeding cover crops after grain crop harvest), broadcast seeding followed by shallow incorporation or rolling, and row crop planters with additional attachments to accommodate small-seeded cover crops.

Aerial or high-clearance seeding equipment may be needed to seed cover crops into tall or normal height standing crops. Aerial seeding using an aircraft modified with a seed disperser can also be used to cost-effectively seed a large number of acres in a timely manner. Manure slurry seeding involves mixing cover crop seed with liquid manure and applying it in the fall. Moisture and nutrients in manure promote rapid cover crop growth, which in turn prevents loss of manure N. It is generally suited for grasses, which are well adapted for establishing quickly and scavenging manure nutrients in the fall.

In certain soil conditions and pest pressures, cover crops can provide habitat for certain corn and soybean pests, including seed corn maggot, wireworm, armyworm, black cutworm, white grubs and slugs. In general, these early season pests are attracted to high residue levels on or in the soil. Early cover crop termination and effective at-planting residue management are the best ways to reduce the risk of pest damage as a result of cover crops.

The field containing maize plants expressing a transgenic yield trait (e.g., increased and extended expression of AG099 or a MADS-box polypeptide) is planted with a cover crop prior to, or during, or after planting of the corn plants. In certain planting systems, the field is planted with a soybean crop prior to planting of the first population of corn plants and then followed by the second crop comprising soybean plants, wherein the first population of corn plants and the second crop of soybean plants are planted in consecutive growing seasons. In certain practices, the cover crop is a perennial cover crop that remains dormant during the crop growing season. The cover crop is planted in a field that is strip tilled and is planted with a cover crop prior to, or during, or after planting of the corn plants.

### Example 5

### Heat Tolerant Maize Plants Containing Increased Expression of AG099

Environmental stresses are responsible for significant yield reduction in agricultural crops. The relation between climate variation and production of corn throughout the United States have been studied and gradual temperature changes have made a measurable negative impact on crop yield.

In some embodiments, the yield trait (e.g., increased and extended expression of AG099) disclosed herein is stacked with one or more additional traits. Thus, the various host cells, plants, plant cells, plant parts, seeds, and/or grain disclosed herein can further comprise one or more traits of interest. In an embodiment, the stress tolerant trait is a heat tolerant trait. As used herein, the term “heat tolerance” is defined as a phenotype where a first plant, or plant line, has increased capacity to withstand elevated temperature and produce a yield that is in excess of a second plant or plant line, the second plant line being a control plant such as a wild-type control plant line. In certain embodiments, the stacked traits comprise at least the yield trait disclosed herein and at least one additional heat tolerance trait. In certain embodiments, the corn plants expressing AG099 display heat tolerance.

These stacked trait combinations can be created by any method including, but not limited to, breeding plants by any conventional methodology, or genetic transformation. The traits can be introduced simultaneously in a co-transformation protocol with the polynucleotides of interest conferring the traits provided by any combination of transformation cassettes. It is further recognized that polynucleotide sequences can be stacked at a desired genomic location using a site-specific recombination system, such as CRISPR-Cas.

The effects of heat stress (HS) can be determined on plants of transgenic lines expressing AG099 compared to a wild-type plant not expressing transgenic AG099. Seeds of wild-type maize and transgenic lines expressing AG099 trait are germinated for 7 days. HS is carried out in growth chambers with ten-day-old seedlings of similar height that are transplanted into pots and are grown under normal temperature, light/dark exposure, and humidity, for example 22° C., 16:8 light:dark cycle and 70% humidity for three more weeks. When the plants are about 5 weeks old, half of WT and transgenic plants are placed under HS conditions, for example 42° C., 16:8 light:dark cycle and 70% humidity, for five days and half of WT and transgenic plants are left at normal temperatures as controls.

Growth parameters in response to HS are evaluated, for example by determining total dry biomass, plant height, stem diameter and chlorophyll content. Growth reduction of transgenic plants expressing AG099 are less affected than WT after five days of heat treatment, with less reduction of dry biomass, plant height, stem diameter, and chlorophyll content compared to WT plants. Similar heat tolerance trait is also assessed in a field environment where the plants are grown in ground soil in a crop growing environment.

### Example 6

### Silage Maize Plants Having Increased and Extended Expression of AG099

Nitrogen promotes rapid growth of corn plants and increases leaf size and quality. Maize plants expressing AG099 are more effective in utilizing nitrogen through its various development cycle. Improved nitrogen availability improves corn yield and its crude protein concentration, which maximizes yields of high quality forage. Transformed maize plants containing the AG099 trait are converted into elite silage inbreds and are crossed for a series of plant biomass yield trials.

A series of trials are conducted in elite silage corn hybrids across testing locations in order to assess the field performance of maize plants (e.g., maize hybrids) and having AG099 events. For example, hybrids with maturities ranging from 105-112 days are used to evaluate the performance of AG099 events relative to controls. AG099 expressing corn hybrids comprise one or more brown midrib mutations to enrich silage quality. Highly digestible, high energy corn silage provides lower dietary undigestible fiber (uNDF240) allowing for higher corn silage inclusion levels, lower supplemental feed costs, improved dry matter intake and potential for higher milk yields. Is is expected that AG099 expressing BMR hybrids to exhibit 5-10, 12 and 15 points higher NDFD30 compared to non-AG099 BMR controls and one ton per acre or more higher silage yield potential compared to control population of plants.

In certain aspects, locations selected to achieve various yield levels can range from highly drought stressed to optimal growing conditions. Soil types include a variety of high sand, sandy loam, silty loam, loam and some clay. In such locations, entries containing the AG099 construct are compared to its control for each specific hybrid background. Two to four replicates of a split plot design are established at each location. A mixed model analysis of variance can be conducted using ASREML where BLUEs (Best Linear Unbiased Estimates) are generated for each AG099 event in combination. Pairwise contrasts of these event BLUEs to wild type BLUEs are analyzed to test significant differences.

In an experimental location, multiple replicates, hybrids with controls, planting densities, and nitrogen (N) treatments are tested to optimize plant biomass yield, for example 4 replicates of 4 hybrids (2 hybrid platforms×2 events) with controls are tested at 5 different planting densities with 2 different nitrogen treatments. The planting densities may range from 20,000 to 44,000 plants per acre.

In another experiment, various applied nitrogen levels were evaluated at different planting densities, for example 34,000 and 44,000 plants per acre.

In another experiment, reduced N rate is evaluated at 5 different planting densities, for example 20000; 26000; 32000; 38000 and 44000, with 4 replicates, two N treatments and 4 hybrids.

In certain aspects, plant biomass yield of silage maize with the AG099 trait are greater than the plant biomass yield of wild-type silage maize. In order to evaluate the response of corn hybrids containing AG099 event across a matrix of planting densities and N treatments provide a prescription of hybrids, N application rates and planting densities to maximize sustainability.

1. A method of optimizing exogenously applied nitrogen use in a field by a population of corn plants, the corn plants comprising a heterologous polynucleotide that encodes a MADS-box polypeptide, the method comprising:
a) providing crop plants wherein the expression of the heterologous polynucleotide increases one or more yield related agronomic parameters to a field that comprises applied nitrogen fertilizer and a nitrogen stabilizer; and
b) increasing the nitrogen assimilation and/or nitrogen utilization rate during the crop plants growing season compared to a control population of plants not comprising the polynucleotide, thereby optimizing the exogenously applied nitrogen in the field.

2. (canceled)

3. The method of claim 1, wherein the corn plants are planted in one or more zones within the field that are characterized by nitrogen run-offs.

4. (canceled)

5. The method of claim 1, wherein the applied nitrogen is used more effectively by the crop plants as measured by a) increase in stalk and/or leaf nitrogen content; b) increase in shoot and/or root biomass; and/or c) increase in root length and/or root density compared to a control population of plants not comprising the trait.

6. (canceled)

7. The method of claim 1, wherein the crop plants are heat tolerant.

8. The method of claim 1, wherein the plants are corn plants that comprise event DP-202216-6.

9. The method of claim 1, wherein the applied nitrogen is about 5% to about 50% less than an application rate of about 50 lbs to about 400 lbs of nitrogen per acre compared to a normal field not comprising the crop plants having the transgenic trait.

10. A method of rotating crops in one or more consecutive growing seasons, the method comprising:
a) growing a population of transgenic corn plants in a first growing season in a field, wherein the expression of a polynucleotide that encodes a MADS-box polypeptide is increased due to a genetic modification, compared to a control plant;
b) growing a population of a second crop plants in a second growing season in the field, wherein the second growing season is consecutive to the first growing season and wherein the second crop plant is not corn; and
c) increasing yield of the population of the soybean plants compared to a control population of soybean plants grown in a control field.

11. The method of claim 11, wherein the corn plants comprise event DP-202216-6.

12. The method of claim 11, wherein the population of second crop plants is selected from the group consisting of soybeans, cotton, wheat and sorghum.

13. The method of claim 11, wherein the increase in yield in the population of the second crop plants is due to increased sequestration of soil organic matter and/or nitrogen.

14. The method of claim 11, wherein the corn plants have early maturity rating and are planted early during the first growing season such that the population of the second crop plants are planted early in the second growing season, the first and second growing seasons are complete within about 8-12 months.

15. The method of claim 11, wherein the field is not tilled, practice conservation tilling, minimum tilling or strip till before planting the corn plants.

16. The method of claim 11, wherein the corn plants are selected to be in a relative maturity zone 80 CRM to about 120 CRM and wherein the second crop plants are soybean plants selected to be in a relative maturity zone of Group 2 to Group 6.

17. The method of claim 11, wherein the field is planted with a cover crop prior to or during or after planting of the corn plants.

18. The method of claim 11, wherein the field is planted with a soybean crop prior to planting of the first population of corn plants and then followed by the second crop comprising soybean plants, wherein the first population of corn plants and the second crop of soybean plants are plant in consecutive growing seasons.

19. (canceled)

20. (canceled)

21. A method of increasing yield by minimizing plant-to-plant variability within a row of a population of corn plants grown in a field, the method comprising:
a) providing a population of corn plants, wherein the expression of a polynucleotide that encodes a polypeptide comprising an amino acid sequence that is at least 90% identical to SEQ ID NO: 1 is increased due to a genetic modification compared to a control plant;
b) planting the corn plants at a density of about 30,000-60,000 plants per acre wherein the row spacing is about 8 inches to about 36 inches; and
c) growing the population of plants in a crop growing environment, wherein the average height variation of the corn plants within a row is less than about 5% to about 20% when measure at a vegetative growth state between V6 to about R1.

22. The method of claim 17, wherein the corn plants comprise event DP-202216-6.

23. The method of claim 17, wherein the corn plants
a) comprise canopy architecture that does not vary by more than about 5% to about 10% when measured by average total surface area of the leaves;
b) exhibit uniform standability when compared to a control population of corn plants;
c) have increased yield due to synchronized pollination and/or increased silk emergence under drought stress; and
d) combinations thereof.

24. (canceled)

25. (canceled)

26. A method for introgressing one or more transgenic insect control traits in corn plants, the method comprising:
a) providing a first female inbred corn plant comprising event DP-202216-6 DNA and a first insect control transgenic trait, wherein the first insect control transgenic trait exhibits reduced inbred yield when the first insect control trait is present in a first population of corn plants in the absence of the event DP-202216-6;
b) providing a first male inbred corn plant comprising a second insect control trait, wherein the second insect control transgenic trait exhibits reduced inbred yield when the second insect control trait is present in a second population of corn plants in the absence of the event DP-202216-6; and
c) crossing the first female inbred plant and the first male corn inbred plant to produce hybrid corn plants comprising the first and second insect control traits and event DP-202216-6.

27. The method of claim 22, wherein the first insect control trait in the first male inbred plant protects the corn plants from one or more lepidopteran pests and wherein the second insect control trait in the first female inbred corn plant protects the corn plants from one or more coleopteran pests.

28.-32. (canceled)
